(19) United States
(12) Patent Application Publication
(10) Pub. No.: US 2021/0063291 A1
(43) Pub. Date: Mar. 4, 2021

(54) FILTER INSTRUMENT, KIT AND METHOD FOR PRETREATING A SAMPLE

(71) Applicant: Boehringer Ingelheim Vetmedica GmbH, Ingelheim am Rhein (DE)

(72) Inventors: Alissa BENTER, Hannover (DE); Dorothee EICKE, Pattensen (DE); Karolin Ursula Luise HORN, Peine (DE); Hannah SCHMOLKE, Didderse (DE); Norbert WENDT, Berlin (DE); Johannes TUCHOLSKI, Berlin (DE)

(73) Assignee: Boehringer Ingelheim Vetmedica GmbH, Ingelheim am Rhein (DE)

(21) Appl. No.: 17/004,040

(22) Filed: Aug. 27, 2020

(30) Foreign Application Priority Data
Aug. 30, 2019 (EP) 19 194 623.5
Apr. 9, 2020 (EP) 20 169 046.8

(51) Int. Cl. G01N 1/40; B01L 3/00; C12N 15/10; G01N 33/53

(52) U.S. Cl. CPC G01N 1/4077; G01N 1/405; B01L 3/502; C12N 15/1017; B01L 2300/069; G01N 33/5306; G01N 2001/4088; B01L 2400/0487; B01L 2300/0681; C12N 15/1006

(57) ABSTRACT

A filter instrument for pretreating, in particular filtering, in particular a biological sample, the filter instrument having a filter arrangement with a sorption filter, a filter element and at least two securing elements at different positions within the housing to hold the filter arrangement. Furthermore, a kit for pretreating in particular a biological sample for an in particular molecular-biological testing of the sample has a plunger with a stop to prevent complete insertion of the plunger into the filter instrument. Furthermore, a method for pretreating an in particular biological sample for an in particular molecular-biological test of the sample within a cartridge in which the plunger is axially secured when/after being inserted in the filter instrument.

### BACKGROUND OF THE INVENTION

### Field of the Invention

The present invention relates to a filter instrument, to a kit and to a method for pretreating an in particular biological sample for, in particular, molecular-biological testing of a sample.

Preferably, the present invention deals with analyzing and/or testing a sample, in particular, from a human or animal, particularly preferably for analytics and diagnostics, for example, with regard to the presence of diseases and/or pathogens and/or for determining blood counts, antibodies, hormones, steroids or the like. Therefore, the present invention is in particular within the field of bioanalytics. A food sample, environmental sample or another sample may optionally also be tested, in particular for environmental analytics or food safety and/or for detecting other substances.

The present invention deals in particular with what are known as point-of-care systems, i.e., in particular with mobile systems, devices and other apparatuses, and deals with methods for carrying out pretreatments and/or tests on a sample at the sampling site and/or independently and/or away from a central laboratory or the like. Preferably, point-of-care systems can be operated autonomously of and/or independently from a mains network for supplying electrical power.

Preferably, at least one analyte or target analyte of a sample can be determined, identified or detected by means of point-of-care systems of this kind. In particular, the sample can be tested for qualitatively or quantitatively determining at least one analyte, for example in order for it to be possible to detect or identify a disease and/or pathogen.

The present invention preferably deals with devices/instruments and other apparatuses for preparing for and/or carrying out a nucleic-acid assay for detecting or identifying a nucleic-acid sequence or a protein assay for detecting or identifying a protein.

### Description of Related Art

U.S. Pat. No. 5,096,669 discloses a point-of-care system for testing a biological sample, in particular a blood sample. The system comprises a single-use cartridge and an analyzer. Once the sample has been received, the cartridge is inserted into the analyzer in order to carry out the test. The cartridge comprises a microfluidic system and a sensor apparatus comprising electrodes, which apparatus is calibrated by means of a calibration liquid and is then used to test the sample.

Furthermore, International Patent Application Publication WO 2006/125767 A1 and corresponding U.S. Pat. No. 10,073,107 B2 disclose a point-of-care system for integrated and automated DNA or protein analysis, comprising a single-use cartridge and an analyzer for fully automatically processing and evaluating molecular-diagnostic analyses using the single-use cartridge. The cartridge is designed to receive a sample, in particular blood, and in particular allows cell disruption, PCR and detection of PCR amplification products, which are bonded to capture molecules and provided with a label enzyme, in order for it to be possible to detect bonded PCR amplification products or nucleic-acid sequences as target analytes in what is known as a redox cycling process.

Once a sample has been taken, the sample usually has to be pretreated/prepared for the subsequent test. For example, in order to test analytes of a sample, it is necessary for the analytes to first be extracted from or dissolved out of the sample, in particular for the analytes to be released/liberated by cell disruption and/or for the sample to be filtered.

International Patent Application Publication WO 2018/234168 A1 and corresponding US Patent Application Publication 2018/0372598 A1 disclose a method and instruments for pretreating a biological sample for a molecular-biological test of the sample, wherein the sample is first lysed within an extraction instrument containing a treatment agent and then dispensed into a cartridge by means of a dispensing instrument that contains an integrated filter for filtering out undesired sample components. Once the pretreated sample has been dispensed into the cartridge, the cartridge is inserted into an analysis device in order to carry out the test.

### SUMMARY OF THE INVENTION

The object of the present invention is to provide an improved instrument, an improved kit and an improved method for pretreating, in particular filtering, of an in particular biological sample for, in particular, molecular-biological testing of a sample, preferably wherein a simple, easy to handle, cost-effective, hygienic and/or rapid pretreatment/preparation of the sample is made possible or facilitated and/or wherein the risk of incorrect use is reduced.

The above-mentioned object is achieved by a filter instrument, by a kit or by a method as disclosed herein.

Within the meaning of the present invention, an “instrument” is preferably a tool, apparatus or device used for receiving, pretreating/preparing, in particular filtering, and/or dispensing a sample which is to be analyzed. Such instruments are particularly preferably manually operated or operable by a user. However, automated or semi-automated solutions are also possible.

In the proposed method, a sample, preferably an oral fluid sample, such as saliva, mucosa or the like, from humans or animals, in particular pigs, is preferably pretreated for a subsequent, in particular molecular-biological test within a cartridge. To this end, the sample is lysed, in particular by means of a treatment/extraction agent, such as a lysis buffer and/or enzymes, and optionally heat treated in order to obtain a lysate. The sample/lysate is then inserted into a filter instrument preferably fluidically connected to a cartridge and, subsequently, pushed through a filter arrangement in the filter instrument, in particular into the cartridge and/or by means of a plunger, in order to filter/purify the sample/lysate, in particular in order to (chromatographically) separate nucleic acids, e.g., DNA and/or RNA, from other biopolymers, e.g. proteins.

Within the meaning of the present invention, analytes are in particular nucleic acid sequences, in particular DNA sequences and/or RNA sequences, or proteins, in particular antigens and/or antibodies. In particular, by means of point-of-care systems, nucleic-acid sequences can be determined/identified or detected as analytes of a sample, or proteins can be determined/identified or detected as analytes of the sample.

According to one aspect of the present invention, the flow of the sample/lysate in the filter arrangement, in particular a sorption filter thereof, is centered, in particular redirected towards the center and/or away from the wall of the filter instrument, mostly preferred by means of a guiding element arranged within the sorption filter. In this way, the contact of the sample/lysate with the filter arrangement, in particular the sorption filter, is increased, in particular such that the filtering/purifying of the sample/lysate is improved.

According to another aspect of the present invention, which can be realized independently, the axial position of the plunger relative to the filter instrument is secured and/or the plunger is axially secured and/or automatically clicked into place after being inserted into the filter instrument and/or when reaching an end position. In this way, the method is facilitated and/or the risk of misapplication is reduced. Further, the plunger and the filter instrument can be disposed as a unit after delivery of the sample/lysate to the cartridge.

The purified sample/lysate can then be tested in or by means of the cartridge, preferably wherein sample components and/or analytes of the sample/lysate, in particular nucleic-acid sequences, are identified or detected, in particular electrochemically.

The sample is preferably delivered or dispensed from the filter instrument directly to the cartridge for testing. In this way, the risk is reduced to contaminate the surroundings and/or the sample.

This allows or facilitates a particularly simple, hygienic and/or reliable pretreatment, in particular lysis, filtering and purification, of the sample.

Within the meaning of the present invention, the term “filter instrument” is preferably understood to mean a structural apparatus/instrument which is designed to pretreat, in particular filter and/or purify, the sample/lysate and/or to separate nucleic acids from other biopolymers within the sample/lysate and/or to introduce the in particular pretreated sample/lysate in a cartridge for the subsequent test of the sample. Mostly preferably, a filter instrument within the meaning of the present invention is embodied as a syringe or the like.

The proposed filter instrument comprises a preferably at least substantially cylindrical housing and a—in particular multilayer—filter arrangement within the housing.

The proposed filter arrangement comprises a (powdery/granular) sorption filter, in particular having a (powdery/granular) chromatographic/sorbent material, such as a (modified/functionalized) silica gel, for (chromatographic) separation/purification of biopolymers, in particular nucleic acids, from other biopolymers and, further, an optional guiding element arranged within, in particular axially enclosed by, the sorption filter in order to guide/redirect, mostly preferred center, the flow of the sample/lysate through the sorption filter.

Due to the optional guiding element, the sample/lysate is guided/redirected towards the center of the sorption filter and/or away from the housing. This prevents that the sample/lysate bypasses the sorption filter. With other words, the contact, i.e., the contact time and/or the contact surface, between the sample/lysate and the sorption filter, in particular its chromatographic/sorbent material, is increased. In this way, the filtration/purification of the sample/lysate is improved.

The guiding element is preferably circular/ring-shaped and/or comprises a preferably centrally arranged passageway, in particular such that the sample/lysate can only pass centrally through the guiding element.

The guiding element is preferably made out of plastic, mostly preferred an elastomer. According to a preferred embodiment, the guiding element is embodied as a sealing ring.

In particular, the guiding element radially and/or sealingly abuts the housing/wall of the filter instrument.

In addition to the sorption filter, the filter instrument, in particular the filter arrangement, comprises preferably at least one, preferably several, filter elements for filtering the sample/lysate, in particular for separating or filtering out undesired sample components, such as particles, impurities, proteins or the like.

By using the proposed filter instrument, it is preferably possible to directly test the pretreated sample/lysate and/or to feed said sample/lysate to the cartridge, such that further preparation or filtration of the sample can be omitted.

Preferably, the filter arrangement is integrated in the filter instrument and/or arranged in the chamber or the housing of the filter instrument. Mostly preferred, the filter arrangement is arranged on or directly upstream of the outlet of the filter instrument, in particular such that the sample is pretreated or filtered in the filter instrument and/or during or immediately before dispensing.

According to another aspect of the present invention, the filter instrument comprises at least two, preferably three, securing elements to (axially) hold/secure/clamp the filter arrangement, in particular different portions thereof, mostly preferred in a form-fitting and/or force-fitting manner, wherein the securing elements are located at different (axial) positions within the housing of the filter instrument. This prevents or at least reduces the risk that the filter arrangement, in particular components thereof, such as the sorption filter or filter elements, get out of place, e.g., during transport and/or when handling the instrument.

Preferably, one of the securing elements, in particular the first securing element, is arranged at a first/upper axial end of the filter arrangement and/or one of the securing elements, in particular the second securing element, is arranged at a second/lower axial end of the filter arrangement.

The proposed kit for pretreating, in particular filtering, in particular a biological sample for in particular molecular-biological testing of the sample comprises the proposed filter instrument for filtering the sample and, further, a—preferably manually operated—plunger to push the sample/lysate through the filter instrument in particular into the cartridge. In this way, corresponding advantages can be achieved.

According to a further aspect of the present invention, which can be realized independently, the plunger comprises a (axial) stop to prevent the complete insertion of the plunger into the filter instrument, in particular to prevent the plunger from abutting/hitting the filter arrangement (axially), and/or to limit the volume pushed through the filter arrangement and/or the pressure generated in the filter instrument by means of the plunger. In this way, the user-friendliness is increased and/or the risk of a misapplication is reduced.

According to a further aspect of the present invention, which can be realized independently, the plunger or the filter instrument comprises a locking part, such as a hook, to axially secure the plunger within the filter instrument and/or to secure the axial position of the plunger relative to the filter instrument, preferably in a form-fit manner and/or by means of a clip-on connection, in particular after the plunger is inserted into the filter instrument and/or when the plunger reaches an end position and/or when the stop of the plunger abuts the filter instrument, in particular its collar.

Preferably, the plunger or the filter instrument comprises a matching part, such as a protrusion, preferably wherein the locking part is adapted to engage and/or snap into the matching part, in particular after the plunger is inserted into the filter instrument and/or when the plunger reaches an end position and/or when the stop of the plunger abuts the filter instrument, in particular its collar, mostly preferred such that the plunger and the filter instrument are connected to one another in a form-fit manner.

Due the locking part and the matching part, the plunger cannot be pulled or pushed out of the filter instrument once the positon of the plunger is secured and/or once the plunger has reached the end position. In this way, the handling of the kit is facilitated and the risk of misapplication is reduced.

Optionally, the kit comprises a container containing the filter instrument in order to protect the filter arrangement, in particular the sorption filter, from drying up.

Mostly preferably, the container, in particular its closure, comprises a humidity buffer that moderates/balances changes in relative humidity within the container. Due to the humidity buffer, the storage stability of the filter arrangement is increased, in particular such that the kit can be used even after a long period of storage.

Within the meaning of the present invention, a kit is in particular a group, a system and/or a combination comprising the filter instrument, the plunger, a receiving instrument for receiving the sample, a lysis tube for extracting the sample, a dispensing instrument and/or preferably dry treatment/extraction agent, in particular a lysis buffer and/or enzymes for lysis of the sample. The filter instrument, the plunger, the receiving instrument, the lysis tube, the dispensing instrument and/or the treatment agent preferably each form a component of the proposed kit.

The components of the kit are preferably marketed as a group, in particular in a common box, container or the like. It is however also possible to market the aforementioned components separately for joint use. Preferably, a common or unifying component is provided in/with the proposed kit, e.g., operating instructions, recommendations for use, references on the labeling of one or more of the components and/or a (common) box, such as a packaging or the like.

Within the meaning of the present invention, a “plunger” is preferably a movable part for the filter instrument which fits tightly, in particular sealingly, into the housing of the filter instrument.

The plunger is preferably manually actuated and/or comprises a handle to manually insert the plunger into the housing in order to push the sample/lysate through the filter arrangement into the cartridge.

In the present invention, the term “lysis tube” is preferably understood to mean a structural apparatus/instrument designed to extract or dissolve components and/or analytes of an in particular biological sample, preferably from a receiving element, such as a swab, and/or to activate said components and/or analytes, in particular by means of a treatment/extraction agent, such as a lysis buffer and/or enzymes.

The above-mentioned aspects, features and steps of the present invention and the aspects, features and steps of the present invention that will become apparent from the following description and can in principle be implemented independently from one another, but also in any combination or order.

Other aspects, advantages, features and properties of the present invention will become apparent from the following description of preferred embodiments with reference to the accompanying drawings.

### BRIEF DESCRIPTION OF THE DRAWINGS

FIG. 1 is a schematic view of an analyzer and a cartridge received in the analyzer;

FIG. 2 is a schematic view of the cartridge;

FIG. 3 is a schematic section through the cartridge when said cartridge is being filled by means of a filter instrument according to a first embodiment of the invention;

FIG. 4 is a schematic view of a proposed kit comprising a lysis tube, a dispensing instrument, a receiving instrument, the filter instrument according to the first embodiment and a plunger;

FIG. 5A is a schematic section through the filter instrument according to the first embodiment;

FIG. 5B is a schematic section through the filter instrument according to a second embodiment of the invention;

FIG. 6A is a schematic view of the lysis tube together with the dispensing instrument when preparing the treatment agent for treatment of a sample;

FIG. 6B is a schematic view of the lysis tube according to FIG. 6A with the receiving instrument being inserted into the lysis tube in order to extract the sample from the receiving instrument;

FIG. 6C is a schematic view of the lysis tube according to FIG. 6B when being inserted into a treatment apparatus for heat treatment of the sample/lysate;

FIG. 6D is a schematic view of the filter instrument according to the first embodiment when being removed out of a container;

FIG. 6E is a schematic view of the cartridge together with the filter instrument fluidically connected thereto when the sample/lysate is filled into the filter instrument by means of the lysis tube;

FIG. 6F is a schematic view of the cartridge together with the filter instrument fluidically connected thereto when the sample/lysate soaks in a filter arrangement of the filter instrument; and

FIG. 6G is a schematic view of the cartridge according to FIG. 6F when the sample is being pushed out of the filter instrument into the cartridge by means of the plunger.

### DETAILED DESCRIPTION OF THE INVENTION

In the figures, which are only schematic and sometimes not to scale, the same reference signs are used for the same or similar parts and components, resulting in corresponding or comparable properties and advantages even if these are not repeatedly described.

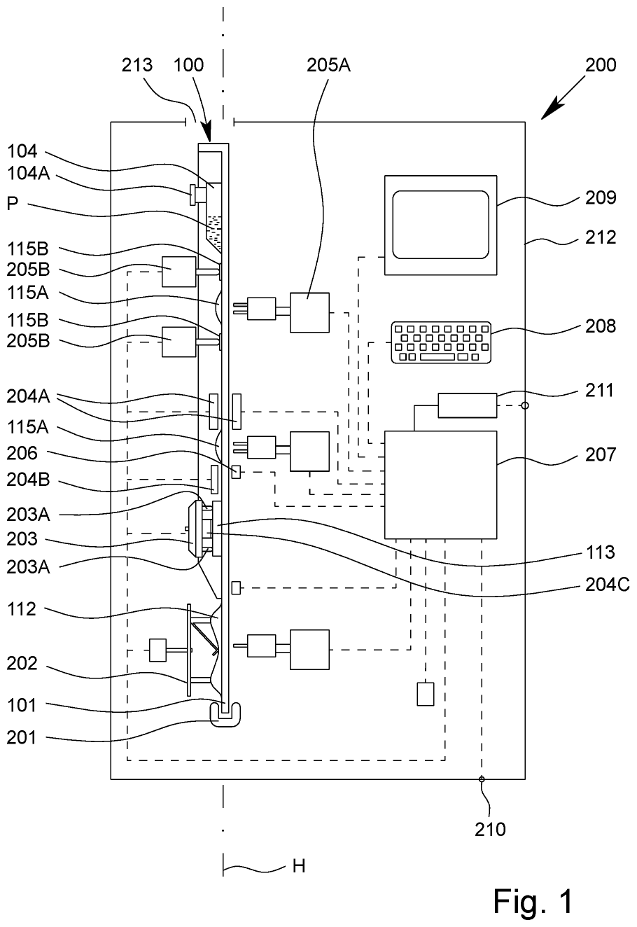

FIG. 1 is a highly schematic view of an apparatus or cartridge 100 in an analysis device or analyzer 200 for an in particular molecular-biological test of an in particular biological sample P.

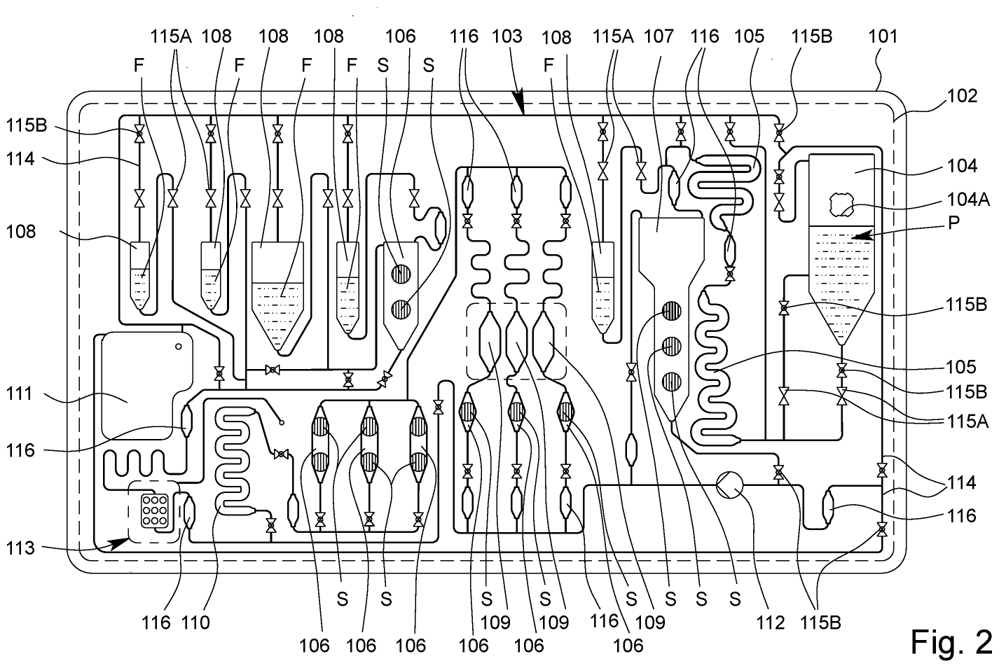

FIG. 2 is a schematic view of a preferred embodiment of the apparatus or cartridge 100 for testing the sample P. The apparatus or cartridge 100 in particular forms a handheld unit, and in the following is merely referred to as a cartridge 100.

The term “cartridge” is preferably understood to mean a structural apparatus or unit designed to receive, to store, to physically, chemically and/or biologically treat and/or prepare and/or to measure a sample, preferably in order to make it possible to detect, identify or determine at least one analyte, in particular a protein and/or a nucleic-acid sequence, of the sample.

A cartridge within the meaning of the present invention preferably comprises a fluid system having a plurality of channels, cavities and/or valves for controlling the flow through the channels and/or cavities.

In particular, within the meaning of the present invention, a cartridge is designed to be at least substantially planar and/or card-shaped, in particular is designed as a (micro)fluidic card and/or is designed as a main body or container that can preferably be closed and/or said cartridge can be inserted and/or plugged into an analyzer when it contains the sample.

The term “analyzer” is preferably understood to mean a structural apparatus designed to chemically, biologically and/or physically test and/or analyze a sample or analysis sample or a component thereof, in particular in order for it to be possible to directly and/or indirectly detect or identify a disease and/or pathogen. An analyzer within the meaning of the present invention is in particular a portable or mobile device designed in particular to immediately or directly test and/or analyze the sample, in particular on site and/or in the vicinity of the sampling site and/or away from a central laboratory.

The term “sample” is preferably understood to mean the sample material to be tested, which is in particular taken from a human or an animal. In particular, within the meaning of the present invention, a sample is a fluid, such as mucosa, saliva, blood, urine or another liquid, preferably from a human or an animal, or a component thereof. Within the meaning of the present invention, a sample may be pretreated or prepared, or may come directly from a human or animal or the like.

A sample within the meaning of the present invention preferably contains one or more sample components or analytes to be tested, it preferably being possible for the analytes to be identified and/or detected, in particular qualitatively and/or quantitatively determined. Particularly preferably, within the meaning of the present invention, a sample has target nucleic acid sequences as the analytes, in particular target DNA sequences and/or target RNA sequences, and/or target proteins as the analytes, in particular target antigens and/or target antibodies. Particularly preferably, at least one disease and/or pathogen can be detected or identified in the sample by qualitatively and/or quantitatively determining the analytes.

Preferably, the analyzer 200 controls the testing of the sample P in particular in or on the cartridge 100 and/or the analyzer 200 is used to evaluate the testing and/or to collect, to process and/or to store measured values from the test.

By means of the analyzer 200 and/or by means of the cartridge 100 and/or using the method for testing the sample P, an analyte of the sample P, or particularly preferably a plurality of analytes of the sample P, can preferably be determined, identified or detected. Said analytes are in particular detected, identified and/or measured not only qualitatively, but particularly preferably also quantitatively.

Therefore, the sample P can be tested for qualitatively or quantitatively determining at least one analyte, for example in order for it to be possible to detect or identify a disease and/or pathogen or to determine other values, which are important for diagnostics, for example.

The cartridge 100 is preferably at least substantially planar, flat, plate-shaped and/or card-shaped.

The cartridge 100 preferably comprises an in particular at least substantially planar, flat, plate-shaped and/or card-shaped main body or support 101, the main body or support 101 in particular being made of and/or injection molded from plastics material, particularly preferably polypropylene.

The cartridge 100 preferably comprises at least one film or cover 102 for covering the main body 101 and/or cavities and/or channels formed therein at least in part, in particular on the front, and/or for forming valves or the like, as shown by dashed lines in FIG. 2.

The cartridge 100 and/or the main body 101 thereof, in particular together with the cover 102, preferably forms and/or comprises a fluidic system 103, referred to in the following as the fluid system 103.

The cartridge 100, the main body 101 and/or the fluid system 103 are preferably at least substantially vertically oriented in the operating position and/or during the test, in particular in the analyzer 200, as shown schematically in FIG. 1. In particular, the main plane H or surface extension of the cartridge 100 thus extends at least substantially vertically in the operating position.

The cartridge 100 and/or the fluid system 103 preferably comprises a plurality of cavities, in particular at least one receiving cavity 104, at least one metering cavity 105, at least one intermediate cavity 106, at least one mixing cavity 107, at least one storage cavity 108, at least one reaction cavity 109, at least one intermediate temperature-control cavity 110 and/or at least one collection cavity 111, the cavities preferably being fluidically interconnected by a plurality of channels.

Within the meaning of the present invention, channels are preferably elongated forms for conducting a fluid in a main flow direction, the forms preferably being closed transversely, in particular perpendicularly, to the main flow direction and/or longitudinal extension, preferably on all sides.

In particular, the main body 101 comprises elongated notches, recesses, depressions or the like, which are closed at the side by the cover 102 and form channels within the meaning of the present invention.

Within the meaning of the present invention, cavities or chambers are preferably formed by recesses, depressions or the like in the cartridge 100 or main body 101, which are closed or covered by the cover 102, in particular at the side. The volume or space enclosed by each cavity is preferably fluidically linked, in particular to the fluid system 103, by means of channels.

In particular, within the meaning of the present invention, a cavity comprises at least two openings for the inflow and/or outflow of fluids.

Within the meaning of the present invention, cavities preferably have a larger diameter and/or flow cross section than channels, preferably by at least a factor of 2, 3 or 4. In principle, however, cavities may in some cases also be elongate, in a similar manner to channels.

The cartridge 100 and/or the fluid system 103 also preferably comprises at least one pump apparatus 112 and/or at least one sensor arrangement or sensor apparatus 113.

The reaction cavity/cavities 109 is/are preferably designed to allow a substance located in the reaction cavity 109 to react when an assay is being carried out, for example by being linked or coupled to apparatuses or modules of the analyzer 200.

The reaction cavity/cavities 109 is/are used in particular to carry out an amplification reaction, in particular PCR, or several, preferably different, amplification reactions, in particular PCRs. It is preferable to carry out several, preferably different, PCRs, i.e. PCRs having different primer combinations or primer pairs, in parallel and/or independently and/or in different reaction cavities 109.

The amplification products, target nucleic acid sequences and/or other portions of the sample P produced in the one or more reaction cavities 109 can be conducted or fed to the connected sensor arrangement or sensor apparatus 113, in particular by means of the pump apparatus 112.

The sensor arrangement or sensor apparatus 113 is used in particular for detecting or identifying, particularly preferably qualitatively and/or quantitatively determining, the analyte or analytes of the sample P, in this case particularly preferably the target nucleic-acid sequences and/or target proteins as the analytes. Alternatively or additionally, however, other values may also be collected or determined.

The cartridge 100, the main body 101 and/or the fluid system 103 preferably comprise a plurality of channels 114 and/or valves 115, in particular initially closed valves 115A and initially open valves 115B, as shown in FIG. 2.

By means of the channels 114 and/or valves 115, the cavities 104 to 111, the pump apparatus 112 and/or the sensor arrangement or sensor apparatus 113 can be temporarily and/or permanently fluidically interconnected and/or fluidically separated from one another, as required and/or optionally or selectively, in particular such that they are controlled by the analyzer 200.

The cavities 104 to 111 are preferably each fluidically linked or interconnected by a plurality of channels 114. Particularly preferably, each cavity is linked or connected by at least two associated channels 114, in order to make it possible for fluid to fill, flow through and/or drain from the respective cavities as required.

The receiving cavity 104 preferably comprises a connection 104A for introducing the sample P. In particular, the sample P may for example be introduced into the receiving cavity 104 and/or cartridge 100 via the connection 104A by means of a pipette, syringe or other instrument, as explained in greater detail in the following.

The cartridge 100 is preferably designed as a microfluidic card and/or the fluid system 103 is preferably designed as a microfluidic system. In the present invention, the term “microfluidic” is preferably understood to mean that the respective volumes of individual cavities, some of the cavities or all of the cavities 104 to 111 and/or channels 114 are, separately or cumulatively, less than 5 ml or 2 ml, particularly preferably less than 1 ml or 800 μl, in particular less than 600 μl or 300 μl, more particularly preferably less than 200 μl or 100 μl.

In particular, a sample P having a maximum volume of 5 ml, 2 ml or 1 ml can be introduced into the cartridge 100 and/or the fluid system 103, in particular the receiving cavity 104.

Reagents and liquids which are preferably introduced or provided before the test in liquid form as liquids or liquid reagents F and/or in dry form as dry reagents S are required for testing the sample P, as shown in the schematic view according to FIG. 2.

Furthermore, other liquids F, in particular in the form of a wash buffer, solvent for dry reagents S and/or a substrate, for example in order to form detection molecules and/or a redox system, are also preferably required for the test, the detection process and/or for other purposes, and are in particular provided in the cartridge 100, i.e. are likewise introduced before use, in particular before delivery.

The cartridge 100 preferably contains all the reagents and liquids required for pretreating the sample P and/or for carrying out the test or assay, in particular for carrying out one or more amplification reactions or PCRs, and therefore, particularly preferably, it is only necessary to receive the optionally pretreated sample P.

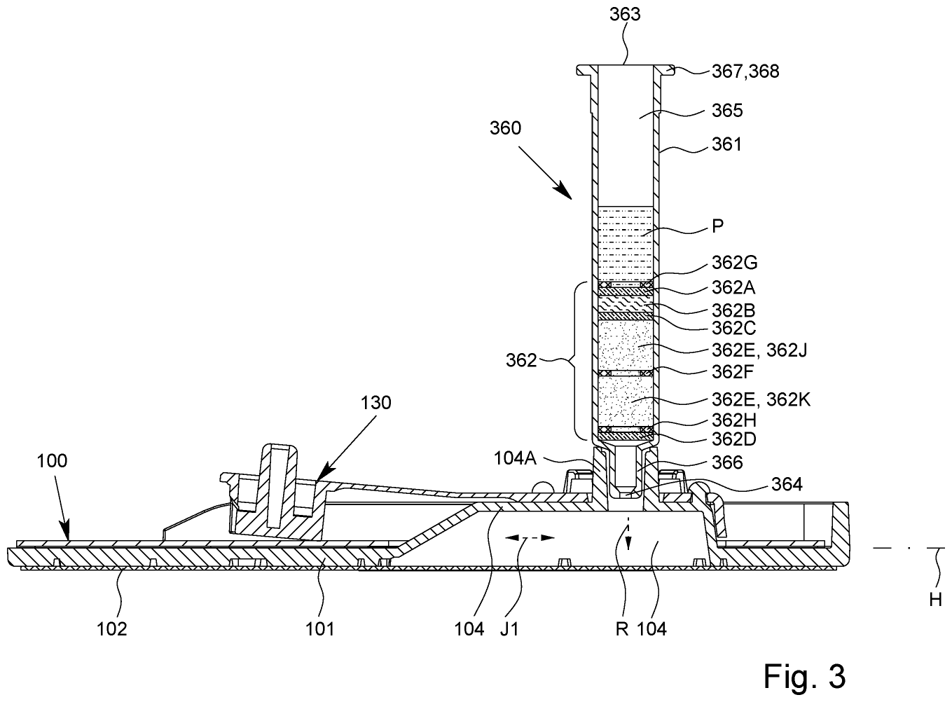

The connection 104A of the receiving cavity 104 can be closed after the sample P has been received. The cartridge 100 preferably comprises a closure element 130 for this purpose, as shown in FIG. 3.

In particular, the connection 104A can be closed in a liquid-tight and particularly preferably also gas-tight manner by the closure element 130. In particular, a closed fluid circuit can thus be formed, with the receiving cavity 104 being included. In particular, once the assigned valves 115A at an inlet, outlet and/or intermediate connection of the receiving cavity 104 have been opened, the receiving cavity 104 thus forms part of the fluid system 103 of the cartridge 100, wherein the fluid system is preferably closed or can be closed by the closure element 130.

The closure element 130 or the closure part thereof closes the receiving cavity 104 or the connection 104A thereof preferably in a permanent manner, i.e. it preferably cannot be released again. The connection 104A therefore preferably cannot be reopened after it has been closed.

FIG. 3 shows the cartridge 100 together with a fluidically connected filter instrument 360, but before the receiving cavity 104 is actually filled with the sample P or before said sample P is actually fed to said cavity 104.

The filter instrument 360 is preferably designed to dispense the pretreated sample/lysate P to the cartridge 100, as explained in greater detail in the following.

In the state shown, the filter instrument 360 is preferably connected to and/or plugged into the connection 104A by means of a connecting tip/piece 366, preferably wherein a vent or slots formed thereby of the connection 104A remain open so that, when the receiving cavity 104 is filled (in part) with the sample/lysate P, gas or air can escape from the receiving cavity 104 to the outside through the vent. In this regard it is noted that, in the delivery state, the valves 115A assigned to the receiving cavity 104 are all closed, and the fluid system 103 is thus closed off from the receiving cavity 104 such that displaced air can escape only through the connection 104A and/or the vent that is particularly preferably provided. However, other structural solutions are in principle also possible.

The main direction R when filling the cartridge 100 with the sample/lysate P is shown schematically in FIG. 3. This main direction R extends in the opposite direction from the main opening direction of the connection 104A.

The main direction R preferably extends transversely and/or perpendicularly to a longitudinal extension J1 of the receiving cavity 104 and/or the main plane H of the cartridge 100.

Specifically, the receiving cavity 104 is designed such that the longitudinal extension J1 thereof extends at least substantially in the vertical direction in the operating position of the cartridge 100.

After the receiving cavity 104 has been filled with the sample P, the filter instrument 360 is removed and the connection 104A is closed by the closure element 130 and/or the closure part thereof is placed onto the connection 104A in order to sealingly or tightly close said connection.

In the closed state, the closure element 130 or the closure part thereof is preferably sealingly or tightly held against or on the connection 104A in a latching, interlocking or form-fitting manner, in the example shown in particular by means of one or more retaining arms or elements which are in particular arm-like and/or which comprise or form one or more latching projections.

Once the sample P has been introduced into the receiving cavity 104 and the connection 104A has been closed, the cartridge 100 can be inserted into and/or received in the proposed analyzer 200 in order to test the sample P, as shown in FIG. 1.

The analyzer 200 preferably comprises a mount or receptacle 201 for mounting and/or receiving the cartridge 100.

Preferably, the cartridge 100 is fluidically, in particular hydraulically, separated or isolated from the analyzer 200. In particular, the cartridge 100 forms a preferably independent and in particular closed or sealed fluidic or hydraulic system 103 for the sample P and the reagents and other liquids. In this way, the analyzer 200 does not come into direct contact with the sample P and can in particular be reused for another test without being disinfected and/or cleaned first.

It is however provided that the analyzer 200 is connected or coupled mechanically, electrically, thermally and/or pneumatically to the cartridge 100.

In particular, the analyzer 200 is designed to have a mechanical effect, in particular for actuating the pump apparatus 112 and/or the valves 115, and/or to have a thermal effect, in particular for temperature-controlling the reaction cavity/cavities 109 and/or the intermediate temperature-control cavity 110.

In addition, the analyzer 200 can preferably be pneumatically connected to the cartridge 100, in particular in order to actuate individual apparatuses, and/or can be electrically connected to the cartridge 100, in particular in order to collect and/or transmit measured values, for example from the sensor apparatus 113 and/or from sensor portions 116.

The analyzer 200 preferably comprises a pump drive 202, the pump drive 202 in particular being designed for mechanically actuating the pump apparatus 112.

The analyzer 200 preferably comprises a connection apparatus 203 for in particular electrically and/or thermally connecting the cartridge 100 and/or the sensor arrangement or sensor apparatus 113.

As shown in FIG. 1, the connection apparatus 203 preferably comprises a plurality of electrical contact elements 203A, the cartridge 100, in particular the sensor arrangement or sensor apparatus 113, preferably being electrically connected or connectable to the analyzer 200 by the contact elements 203A.

The analyzer 200 preferably comprises one or more temperature-control apparatuses 204 for temperature-controlling the cartridge 100 and/or having a thermal effect on the cartridge 100, in particular for heating and/or cooling, the temperature-control apparatus(es) 204 (each) preferably comprising or being formed by a heating resistor or a Peltier element.

Preferably, individual temperature-control apparatuses 204, some of these apparatuses or all of these apparatuses can be positioned against the cartridge 100, the main body 101, the cover 102, the sensor arrangement, sensor apparatus 113 and/or individual cavities and/or can be thermally coupled thereto and/or can be integrated therein and/or can be operated or controlled in particular electrically by the analyzer 200. In the example shown, in particular the temperature-control apparatuses 204A, 204B and/or 204C are provided.

The analyzer 200 preferably comprises one or more actuators 205 for actuating the valves 115. Particularly preferably, different (types or groups of) actuators 205A and 205B are provided which are assigned to the different (types or groups of) valves 115A and 115B for actuating each of said valves, respectively.

The analyzer 200 preferably comprises one or more sensors 206. In particular, sensors 206 are assigned to the sensor portions 116 and/or are designed or provided for detecting liquid fronts and/or flows of fluid in the fluid system 103, the ambient temperature, internal temperature, atmospheric humidity, position, and/or alignment, for example by means of a GPS sensor, and/or the orientation and/or inclination of the analyzer 200 and/or the cartridge 100.

The analyzer 200 preferably comprises a control apparatus 207, in particular comprising an internal clock or time base for controlling the sequence of a test or assay and/or for collecting, evaluating and/or outputting or providing measured values in particular from the sensor apparatus 113, and/or from test results and/or other data or values.

The control apparatus 207 preferably controls or feedback controls the pump drive 202, the temperature-control apparatuses 204 and/or actuators 205, in particular taking into account or depending on the desired test and/or measured values from the sensor arrangement or sensor apparatus 113 and/or sensors 206.

Optionally, the analyzer 200 comprises an input apparatus 208, such as a keyboard, a touch screen or the like, and/or a display apparatus 209, such as a screen.

The analyzer 200 preferably comprises at least one interface 210, for example for controlling, for communicating and/or for outputting measured data or test results and/or for linking to other devices, such as a printer, an external power supply or the like. This may in particular be a wired or wireless interface 210.

The analyzer 200 preferably comprises a power supply 211 for providing electrical power, preferably a battery or an accumulator, which is in particular integrated and/or externally connected or connectable.

The analyzer 200 preferably comprises a housing 212, all the components and/or some or all of the apparatuses preferably being integrated in the housing 212. Particularly preferably, the cartridge 100 can be inserted or slid into the housing 212, and/or can be received by the analyzer 200, through an opening 213 which can in particular be closed, such as a slot or the like.

The analyzer 200 is preferably portable or mobile. Particularly preferably, the analyzer 200 weighs less than 25 kg or 20 kg, particularly preferably less than 15 kg or 10 kg, in particular less than 9 kg or 6 kg.

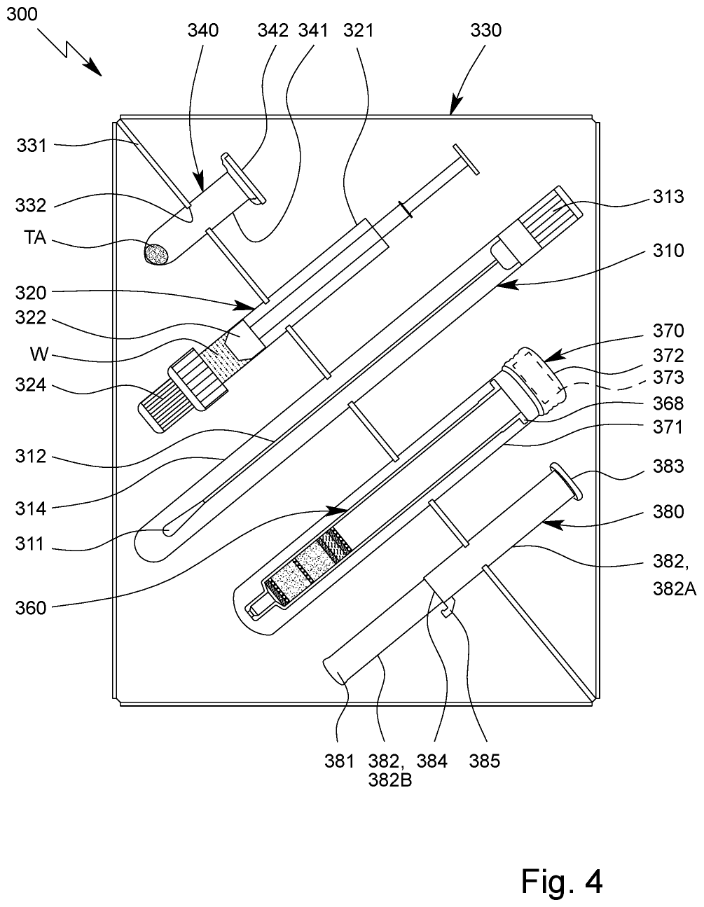

In the following, the pretreatment and/or preparation of the sample P for the test by means of the cartridge 100 and the analyzer 200 will be described, with reference to FIG. 4 to FIG. 6G.

FIG. 4 shows a proposed kit 300 for pretreating the sample P for the test by means of the above-described cartridge 100 and/or the analyzer 200.

The kit 300 preferably comprises a receiving instrument 310, a dispensing instrument 320, a box 330, a lysis tube 340, the filter instrument 360, a container 370 for the filter instrument 360 and/or a plunger 380, the receiving instrument 310, the dispensing instrument 320, the lysis tube 340, the filter instrument 360, the container 370 and/or the plunger 380 preferably being arranged, marketed and/or transported as a group and/or in the (common) box 330.

The kit 300 optionally comprises a treatment/extraction agent TA, preferably wherein the treatment agent TA comprises or is a lysis buffer and/or comprises enzymes for lysis of the sample P.

The treatment agent TA or a component thereof is preferably already contained in the dispensing instrument 320 and/or lysis tube 340 in the delivery state of the kit 300.

In the present embodiment, the lysis tube 340 contains the treatment agent TA, preferably in a dry form and/or as a pellet.

Preferably, the dispensing instrument 320 comprises a solvent W, such as water, to dissolve the (dry) treatment agent TA.

However, it is also possible that the dispensing instrument 320 or the lysis tube 340 comprises the ready-to-use treatment agent TA, i.e. in liquid form.

The box 330 is preferably designed as a packaging, a case or the like. Preferably, the box 330 is at least essentially cuboid.

The box 330 preferably comprises a mount 331 for the components of the kit 300, in particular the receiving instrument 310, the dispensing instrument 320, the lysis tube 340, the filter instrument 360, the container 370 and/or the plunger 380. Preferably, the components of the kit 300, i.e., the receiving instrument 310, the dispensing instrument 320, the lysis tube 340, the filter instrument 360, the container 370 and/or the plunger 380, are held in the box 330 by means of the mount 331 in a form-fitting and/or force-fitting manner or such that they are spaced apart from each other and/or immovably relative to one another.

The box 330, in particular the mount 331, preferably comprises a plurality of receiving portions 332, a separate receiving portion 332 preferably being provided for each component of the kit 300.

Preferably, the mount 331 and/or the receiving portions 332 is/are adapted to the geometry and/or size of the components of the kit 300, in particular such that the components are held at least substantially without any play, clearance or backlash and/or in a form-fitting and/or force-fitting manner.

In the embodiment shown, the mount 331 is formed by a diagonally extending raised portion or rib in the box 330, the receiving portions 332 preferably being formed by concave or at least substantially circular cut-outs in the mount 331. However, other structural solutions are also possible here, for example, wherein the mount 331 and/or the receiving portions 332 are formed by loops, hooks, brackets and/or depressions in a flexible material, such as foam.

The mount 331 and/or the receiving portions 332 allow a particularly space-saving arrangement of the components in the box 330.

Preferably, the components of the kit 300 are arranged in the box 330 obliquely to the long sides of the box 330 and/or such that the longitudinal axes of the components are arranged at least substantially orthogonally to an (imaginary) diagonal of the box 330 and/or the mount 331.

Particularly preferably, the longer components, in this case the receiving instrument 310, the dispensing instrument 320 and the filter instrument 360, are arranged centrally in the box 330, and/or the shorter components, in this case the lysis tube 340 and the plunger 380, are arranged outwardly or in the edge region of the box 330.

The receiving instrument 310 is preferably designed to remove/receive a sample P from a human or animal, in particular a pig, and/or to provide a received sample P for the pretreatment of the sample P.

Preferably, the receiving instrument 310 comprises a receiving element 311, such as a swab, in particular a cotton swab, a connection element 312, in particular in the form of a thin rod, and/or a holding element or a handle 313, the receiving element 311 and the holding element 313 preferably being arranged at opposite axial ends of the receiving instrument 310 and/or being interconnected by means of the connection element 312.

Preferably, the receiving instrument 310 is elongate and/or comprises a length of more than 3 cm or 10 cm and/or less than 100 cm, 80 cm or 30 cm.

By means of the receiving instrument 310, a user of the kit 300 or receiving instrument 310 is able to remove the sample P and/or sample material containing the sample P from a human or animal, in particular a pig, in particular without thereby coming into (direct) contact with the sample material or the human or animal.

Preferably, the receiving instrument 310 comprises an optional vessel 314, the receiving element 311 and/or the connection element 312 preferably being arranged inside the vessel 314 in the delivery state, and/or the holding element 313 forming or comprising a closure element or a cap of the vessel 314.

The dispensing instrument 320 is preferably designed to introduce a fluid, in particular the treatment agent TA, a component thereof and/or the solvent W, into the lysis tube 340, in particular in order to prepare, mostly preferred dissolve, the (dry) treatment agent TA for the lysis of the sample P.

Preferably, the dispensing instrument 320 is designed as a syringe and/or comprises a preferably cylindrical housing 321, a piston 322, a preferably conical connection 323 (not shown in FIG. 4) and/or a closure element 324, the piston 322 preferably being arranged so as to be axially movable in the housing 321, and/or it being possible for a fluid, here the solvent W, to be dispensed via the connection 323 by actuating the dispensing instrument 320 or the piston 322. However, the dispensing instrument 320 might also be embodied as a pipette, a bottle or the like.

As already explained, the treatment agent TA or a component thereof is preferably contained in the lysis tube 340 in the delivery state of the kit 300/lysis tube 340, such that the treatment agent TA can be prepared for lysis, in particular dissolved and/or activated, by transferring the solvent W directly into the lysis tube 340, preferably by means of the dispensing instrument 320.

The lysis tube 340 is preferably designed to pretreat the sample P. In particular, the sample P can be pretreated or prepared, in particular lysed/disrupted, for the test of the sample P by means of the lysis tube 340 and/or in the lysis tube 340.

The lysis tube 340 is preferably designed to receive the receiving instrument 310 or the receiving element 311 at least in part. In particular, the receiving element 311 can be introduced at least in part into the lysis tube 340, in particular in order to pretreat the sample P received by means of the receiving element 311, particularly preferably in order for the analytes of the sample P to be dissolved out of or extracted from said sample P and/or activated/lysed in particular by cell disruption.

The lysis tube 340 preferably comprises a main body 341 and a cap 342, the main body 341 and the cap 342 forming a preferably sealable receptacle for the treatment agent TA, the sample P and/or the receiving element 311.

Preferably, the lysis tube 340 is formed integrally, in particular wherein the main body 341 and the cap 342 are made of and/or injection molded from plastic.

In the present embodiment, the cap 342 is connected to the main body 341 by means of a film hinge. However, the cap 342 might also be a separate part, such as a screw cap.

As already mentioned, the filter instrument 360 is preferably arranged within the container 370, at least in the delivery state of the kit 300.

The container 370 preferably contains the filter instrument 360 in a gas-tight manner and/or such that the filter instrument 360 is protected from drying out.

The container 370 preferably comprises a preferably elongated main body 371 and a closure 372, the main body 371 and the closure 372 forming a preferably sealed receptacle for the filter instrument 360.

The container 370, in particular the main body 371, is preferably embodied as an elongated tube that is open at the top and closed at its bottom.

Preferably, the container 370, in particular its main body 371, is made out of glass, metal, in particular aluminum, or plastic.

As indicated by dashed lines, the container 370 preferably comprises a humidity buffer 373 in order to moderate/balance changes in relative humidity within the container 370, e.g. caused by a temperature change during storage or transportation of the kit 300.

Preferably, the humidity buffer 373 is arranged within and/or attached to the closure 372.

Mostly preferred, the humidity buffer 373 comprises or consists of silica gel.

The relative humidity within the container 370 is preferably greater than 50% or 60%, in particular greater than 70%, and/or less than 95% or 90%, in particular less than 80%, mostly preferred at a temperature of 25° C.

In particular due to the humidity buffer 373, the relative humidity within the container 370 remains in the previously mentioned range, i.e. within the range of 50% to 95%, mostly preferred within the range of 60% to 80%, even if the temperature within the container 370 varies between 0° C. and 35° C. or between 10° C. and 30° C.

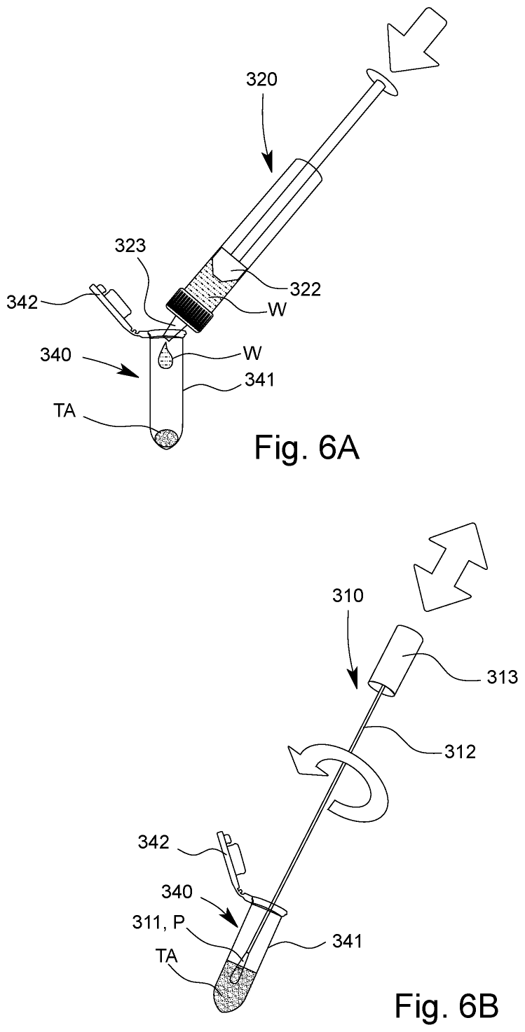

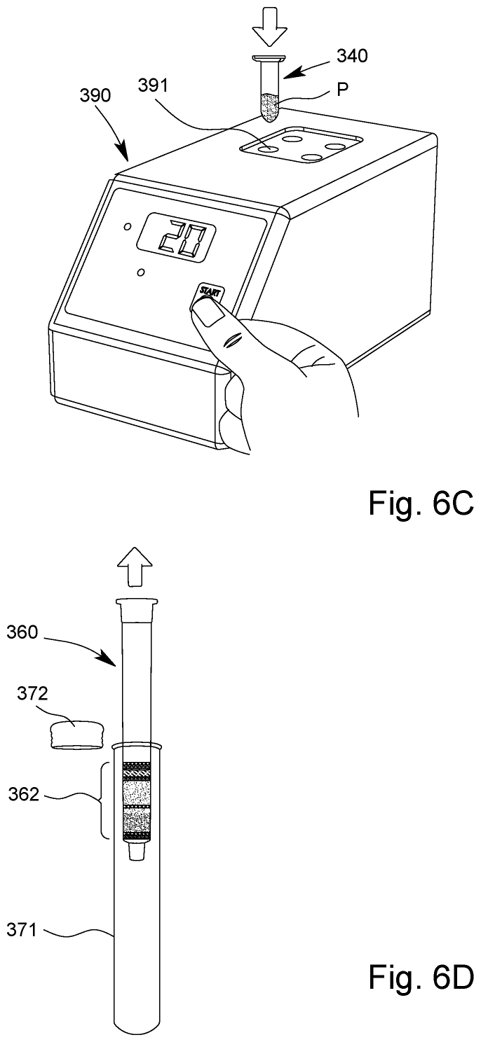

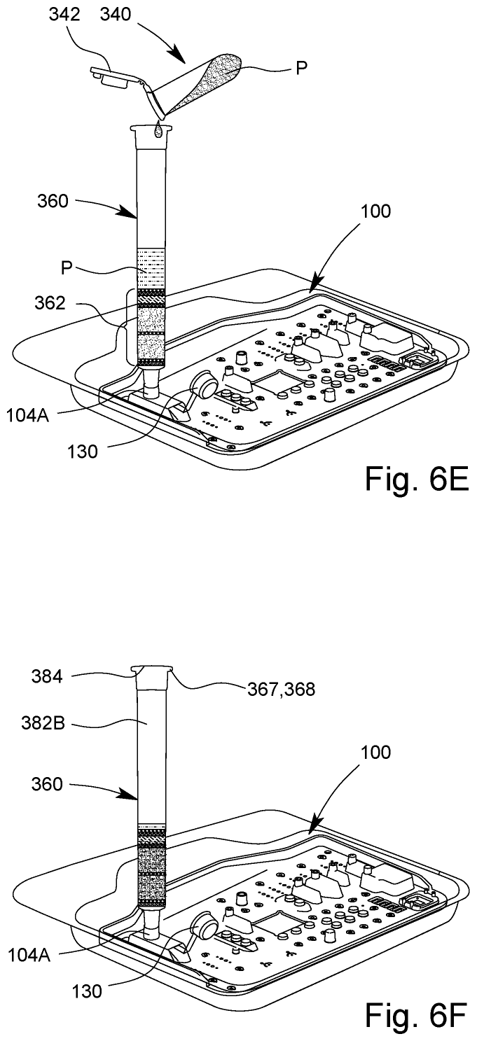

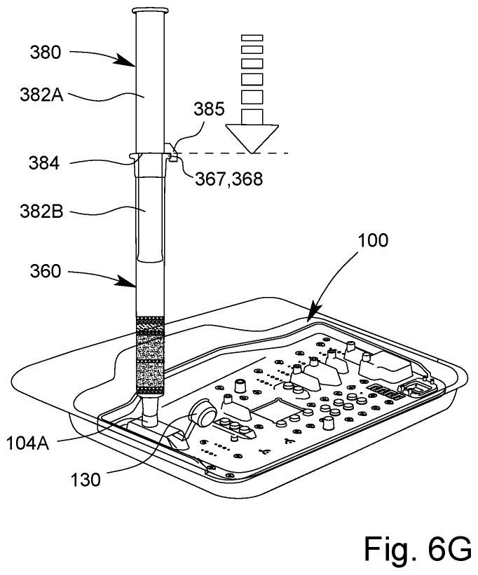

The plunger 380 is preferably adapted to be used together with the filter instrument 360, as will be explained in further detail with reference to the FIGS. 6A to 6G.

Here, the plunger 380 is held within the box 330 separately. However, it is also possible to arrange the plunger 380 in the filter instrument 360 and/or the container 370 in the delivery state of the kit 300.

The plunger 380 is preferably embodied as an elongated and/or at least cylindrical tool.

The plunger 380 is preferably formed in one piece/integrally, preferably wherein the plunger 380 is made out of and/or injection-molded from plastic.

Preferably, the plunger 380 is adapted to be at least partially inserted into the filter instrument 360.

The plunger 380 preferably comprises a preferably mushroom shaped head 381, a shaft 382 and a preferably collar-like handle 383, preferably wherein the head 381 and the handle 383 are connected to the shaft 382 on opposite ends of the shaft 382.

The (outer) diameter of the plunger 380, in particular its shaft 382, preferably changes along the longitudinal axis of the plunger 380.

The plunger 380, in particular the shaft 382, preferably comprises a first portion 382A and a second portion 382B, preferably wherein the first portion 382A comprises an outer diameter that is larger than the outer diameter of the second portion 382B, in particular by at least 1 mm and/or at most 10 mm.

Preferably, the head 381 is attached to and/or formed by the second portion 382B.

Preferably, the handle 383 is attached to and/or formed by the first portion 382A.

The plunger 380, in particular (only) its head 381/second portion 382B, can be inserted into the filter instrument 360.

Preferably, the outer diameter of the first portion 382A is larger than the inner diameter of the filter instrument 360 and/or the outer diameter of the head 381/second portion 382B is smaller than the inner diameter of the filter instrument 360.

The (largest) outer diameter of the head 381 is preferably larger than the outer diameter of the second portion 382B of the shaft 382. Preferably, the (largest) outer diameter of the head 381 corresponds at least essentially to the inner diameter of the filter instrument 360.

It is preferred that the head 381 sealingly abuts the inner wall of the filter instrument 360, in particular such that no fluid can leak out when inserting the plunger 380 into the filter instrument 360.

The plunger 380 preferably comprises a stop 384 to prevent complete insertion of the plunger 380 into the filter instrument 360.

The stop 384 is preferably formed by the transition between the first portion 382A and the second portion 382B.

Mostly preferred, the stop 384 is formed by a step in the transition between the first portion 382A and the second portion 382B. However, it is also possible, that the shaft 382 is tapered in the direction of the head 381, in particular evenly and/or conically.

Preferably, the plunger 380 reaches an end position when the stop 384 (axially) abuts the filter instrument 360, in particular its axial end.

Preferably, the plunger 380 and the filter instrument 360 can be connected to one another in a form-fitting manner and/or such that the axial position of the plunger 380 relative to the filter instrument 360 is secured after insertion of the plunger 380 into the filter instrument 360.

Preferably, the plunger 380 or the filter instrument 360 comprises a locking part 385 to secure the plunger 380 axially in the filter instrument 360.

In the present embodiment, the plunger 380 comprises the locking part 385. However, it is also possible that the filter instrument 360 comprises the locking part 385 and/or an additional locking part (not shown).

The locking part 385 is preferably formed by the transition between the first portion 382A and the second portion 382B. Preferably, the locking part 385 is positioned directly next to the stop 384 and/or comprises or forms the stop 384.

Preferably, the locking part 385 is embodied as a (snap-in) hook/catch/latch, in particular wherein the hook/catch/latch projects from the first portion 382A towards the second portion 382B and/or extends beyond the stop 384.

Preferably, the locking part 385 is curved in a circumferential direction of the plunger 380 and/or according to the (outer) diameter of the filter instrument 360.

In the present embodiment, the plunger 380 comprises a single locking part 385. However, the plunger 380 or the filter instrument 360 might also be equipped with a plurality of locking parts 385, in particular being (evenly) distributed over the circumference of the plunger 380 or the filter instrument 360.

The filter instrument 360, in particular its axial end, preferably comprises a matching part 368, preferably wherein the locking part 385 is adapted to engage the matching part 368, in particular in a form-fit manner and/or after the plunger 380 is inserted into the filter instrument 360 and/or when the plunger 380 reaches the end position.

In the present embodiment, the filter instrument 360 comprises the matching part 368. However, it is also possible that the plunger 380 comprises the matching part 368 and/or an additional matching part (not shown).

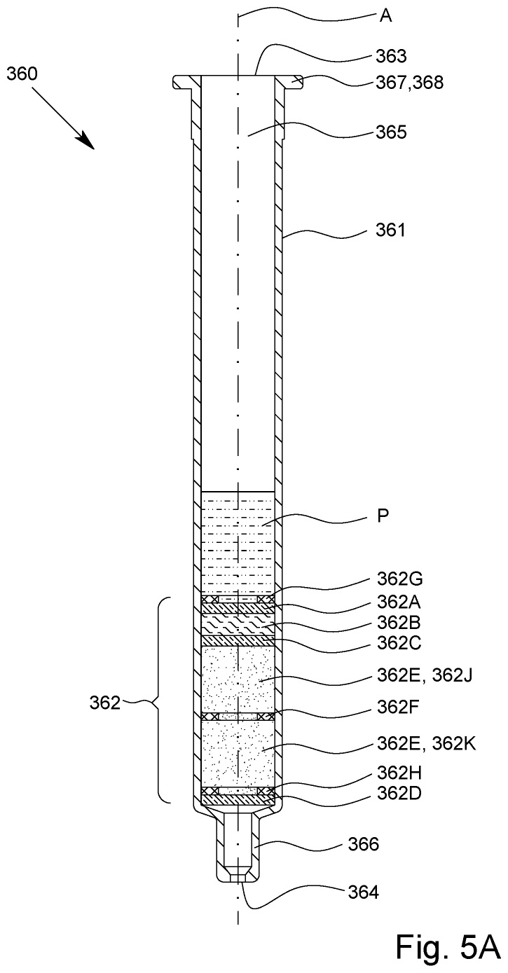

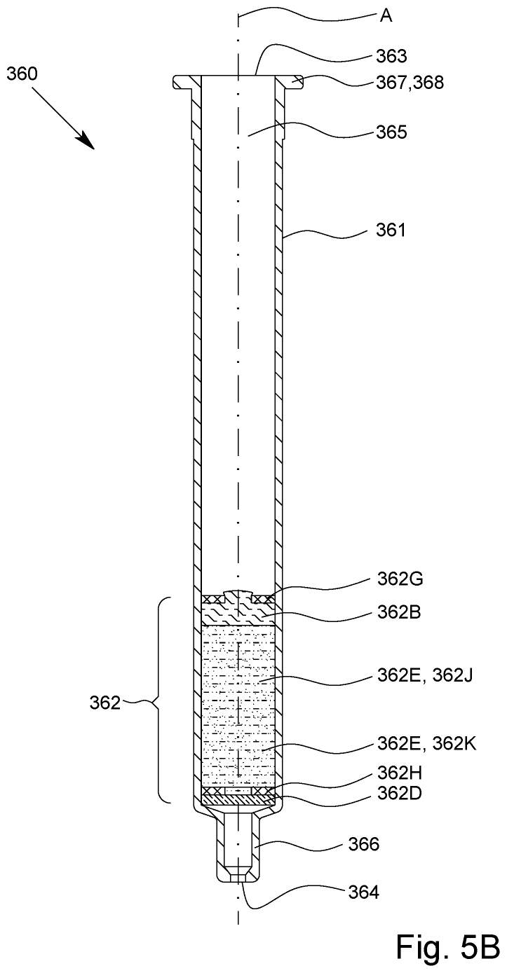

FIG. 5A is a schematic section through the proposed filter instrument 360 according to a first embodiment.

The filter instrument 360 is preferably designed for (further) pretreatment or preparation, in particular filtration and/or purification, of the sample/lysate P that has in particular already been pretreated or prepared via the lysis tube 340 and/or a treatment apparatus 390 (not shown).

Preferably, the filter instrument 360 is designed to (directly) dispense the pretreated sample/lysate P to the cartridge 100, the sample P preferably being (further) prepared or pretreated, in particular filtered/purified, by means of or during dispensing of the sample/lysate P, as described in greater detail in the following.

Preferably, the filter instrument 360 is, in principle, constructed as a housing of a syringe.

Preferably, the filter instrument 360 comprises a housing 361 and a filter arrangement 362, wherein the filter arrangement 362 is arranged within the housing 361.

The filter instrument 360, in particular its housing 361, is preferably elongated, cylindrical and/or rotationally symmetric. Mostly preferred, the filter instrument 360, in particular the housing 361, is embodied as a hollow cylinder.

Preferably, the filter instrument 360, in particular its housing 361, comprises or defines an axis A, in particular wherein the axis A is a central, longitudinal and/or rotation axis of the filter instrument 360.

In the following, spatial orientations and/or arrangements, in particular the terms “radial” or “radially” and/or “axial” or “axially”, refer to the axis A, unless otherwise stated.

The filter instrument 360 preferably comprises an inlet 363, an outlet 364 and/or a chamber 365, the inlet 363 preferably being fluidically connected to the outlet 364 by means of the chamber 365.

The inlet 363 and the outlet 364 are preferably arranged at opposite axial ends of the housing 361.

The filter instrument 360 preferably comprises a (tapered) connecting piece/tip 366, preferably wherein the connecting piece 366 comprises or forms the outlet 364.

Preferably, the inner diameter of the connecting piece 366 is smaller than the inner diameter of the housing 361.

Preferably, the filter instrument 360, in particular the housing 361, comprises a holder/collar 367 preferably for holding the filter instrument 360, in particular when the filter instrument 360 is actuated, the holder 367 preferably being formed by a (circumferential) collar or two opposing radial projections at the axial end of the housing 361 that comprises or forms the inlet 363 of the filter instrument 360.

As already mentioned, the filter instrument 360, in particular the housing 361, comprises or forms the matching part 368 for the plunger 380, in particular its locking part 385. Preferably, a projection, in particular the holder/collar 367 of the filter instrument 360, comprises or forms the matching part 386. However, the matching part 386 might also be formed by a recess, such as a groove, slot, notch or the like, in particular in the housing 361.

The locking part 385 is preferably adapted to engage behind and/or snap/click into the matching part 386, in particular formed by the holder/collar 367.

The filter arrangement 362 is preferably designed to filter/purify the sample/lysate P, in particular to remove undesired sample components, such as particles, impurities, proteins or the like, and/or to let through (only) nucleic-acids, mostly preferred DNA and/or RNA, as analytes of the sample/lysate P.

Particularly preferably, the filter arrangement 362 is arranged on or directly upstream of the outlet 364 and/or the connecting piece 366, in particular such that the sample/lysate P flows through the filter arrangement 362 or is filtered/purified by means of the filter arrangement 362 immediately before dispensing.

The filter arrangement 362 comprises a plurality of different elements/components that are stacked on top of each other and/or arranged in sequence and/or one behind one another, in particular in the axial direction of the filter instrument 360.

The filter arrangement 362 preferably comprises a plurality of filter elements, in particular a (first) filter element 362A, a (second) filter element 362B, a (third) filter element 362C and/or a (fourth) filter element 362D, a sorption filter 362E, a guiding element 362F and/or a plurality of securing elements, in particular a first securing element 362G and a second securing element 362H.

The filter element(s) 362A-362D is/are preferably adapted to filter out (larger) particles, impurities or the like, in particular having an average diameter of more than 10 μm, 50 μm or 10 μm.

The (first) filter element 362A, the (third) filter element 362C and/or the (fourth) filter element 362D are/is preferably embodied as a porous filter, in particular made out of glass or ceramic, mostly preferred by sintering together glass and/or ceramic particles. Preferably, the (first) filter element 362A, the (third) filter element 362C and/or the (fourth) filter element 362D are/is embodied as a preferably plate-shaped frit.

The filter elements 362A-362D, in particular the (first) filter element 362A, the (third) filter element 362C and the (fourth) filter element 362D, are preferably constructed equally.

The (second) filter element 362B preferably comprises or consists of cotton, mostly preferred made out of synthetic fiber, such as polyester.

The (second) filter element 362B preferably helps to moderate changes in the relative humidity within the container 370 and/or to prevent the filter arrangement 362 from drying out.

Preferably, the (second) filter element 362B is adapted to soak the sample/lysate P in the sorption filter 362E, as will be explained further below.

Preferably, the filter element(s) 362A-362D comprise(s) a thickness of more than 1 mm or 1.2 mm and/or less than 2 mm or 1.6 mm and/or a diameter of more than 5 mm or 7 mm and/or less than 15 mm or 12 mm.

Preferably, the diameter of the filter element(s) 362A-362D are/is (slightly) larger than the inner diameter of the housing 361, preferably by more than 0.05 mm and/or less than 0.15 mm, in particular by at least essentially 0.1 mm.

The filter element(s) 362A-362D are/is preferably press-fitted into the housing 361, in particular such that the sample/lysate P cannot bypass the filter element(s) 362A-362D.

The filter element(s) 362A-362D might comprise the same or similar (average) pore size, e.g., within the range of 1 μm to 100 μm. However, it is preferred that the (average) pore size of the first filter element 362A is larger than the average pore size of the third filter element 362C and/or the fourth filter element 362D.

When being pushed through the filter instrument 360, in particular the filter arrangement 362, the sample/lysate P preferably flows axially through the filter instrument 360, in particular the filter arrangement 362, i.e., towards the outlet 364.

Thus, the main flow direction goes from the inlet 363 towards the outlet 364 along the axis A and/or preferably extends parallel to the axis A.

The (first) filter element 362A is preferably the outermost filter element and/or the filter element which comes first into contact with the sample/lysate P when the sample/lysate P is pushed through the filter arrangement 362. However, other constructions are possible as well.

The (first) filter element 362A is preferably arranged at an axial end of the filter arrangement 362 and/or at the side of the filter arrangement 362 facing away from the outlet 364 and/or connecting piece 366 and/or towards the inlet 363.

The (second) filter element 362B is preferably arranged (directly) downstream of the (first) filter element 362A and/or (directly) upstream of the (third) filter element 362C. With other words, the (second) filter element 362B is preferably arranged/sandwiched between the (first) filter element 362A and the (third) filter element 362C.

The (third) filter element 362C is preferably arranged (directly) downstream of the (second) filter element 362B and/or (directly) upstream of the sorption filter 362E.

The sorption filter 362E is preferably arranged (directly) downstream of (second) filter element 362B and/or the (third) filter element 362C.

The sorption filter 362E is preferably adapted to—in particular chromatographically—separate nucleic acids, in particular DNA and/or RNA, from other biopolymers, in particular proteins.

Preferably, the sorption filter 362E is adapted to bind/restrain proteins and/or to let through nucleic acids, in particular DNA and/or RNA.

With other words, the sorption filter 362E preferably has a high affinity/binding capacity to proteins and/or only low/minimal non-specific interaction with and/or sorption/retention of nucleic acids, in particular DNA and/or RNA.

The sorption filter 362E preferably comprises or consists of a chromatographic/sorbent material, such as silica gel (surface-modified), and in particular has an average pore size of more than 1 nm or 2 nm and/or less than 20 nm, 10 nm and/or 5 nm.

The chromatographic/sorbent material, in particular the silica gel, preferably comprises a copolymer coating, preferably wherein the copolymer coating is hydrophobic, lipophilic and/or preferably comprises polar groups, e.g. carboxyl groups, or negative charges.

Mostly preferred, the chromatographic/sorbent material comprises a hydrophobic coating which is further modified with unsaturated and/or aromatic hydrocarbons, in particular containing polar and/or ionizable functional groups. In particular, unsaturated and/or aromatic copolymers preferably with polar and/or ionizable functional groups may be used.

Preferably, the free end of the hydrocarbons, in particular the copolymer, comprises a carboxyl group, i.e. in the form of a carboxylate or a carboxylic acid ester.

In this way, the interaction with/retention of the predominantly hydrophobic proteins is increased while nucleic acids, in particular DNA and/or RNA, are largely repulsed.

Mostly preferred, the chromatographic/sorbent material of the sorption filter 362E is structured as disclosed in para. [0012] of European Patent EP 1 756 178 B1 and in column 2, lines 38-53 of corresponding U.S. Pat. No. 10,221,411 B2.

The sorption filter 362E, in particular its chromatographic/sorbent material, is preferably a bulk/granular/powdery material.

Preferably, the sorption filter 362E comprises a volume of more than 0.5 ml, in particular of more than 1 ml, mostly preferred of more than 1.5 ml, and/or of less than 5 ml or 4 ml, in particular less than 2.5 ml, mostly preferred of less than 2 ml.

The (fourth) filter element 362D is preferably arranged (directly) downstream of the sorption filter 362E and/or (directly) upstream of the connecting piece 366 and/or the outlet 364.

Mostly preferred, the (fourth) filter element 364D is arranged on a side/end of the filter arrangement 362 facing the connecting piece 366 and/or the outlet 364.

Preferably, the filter elements 362A-362D, in particular the (second) filter element 362B, the (third) filter element 362C and (the) fourth filter element 362D, prevent that the sorption filter 362E, in particular its chromatographic/sorbent material, falls out of the filter instrument 360.

The sorption filter 362E, in particular the chromatographic/sorbent material, is preferably arranged/sandwiched/enclosed between/by the filter elements 362A-362D, in particular the (second) filter element 362B and/or the (third) filter element 362C on the one side and the (fourth) filter element 362D on the other side.

With other words, the filter elements 362A-362D, in particular the (second) filter element 362B, the (third) filter element 362C and/or the fourth filter element 362D, preferably hold the sorption filter 362E in position within the filter instrument 360 and/or prevent falling apart of the preferably granular/powdery sorption filter 362E.

Preferably, the (fourth) filter element 362D (directly) abuts a shoulder formed by the transition between the housing 361 and the connecting piece 366.

The aforementioned volume of the chromatographic/sorbent material for the sorption filter 362E is preferred in order to obtain a sufficiently filtered/purified sample/lysate P for the subsequent test of the sample/lysate P.

However, it has been noted that a non-negligible quantity of the sample/lysate P might flow along the surface of the housing 361 and/or between the housing 361 and the sorption filter 362E, thereby reducing the contact between the lysate/sample P and the sorption filter 362E, in particular its chromatographic/sorbent material.

In particular in order to increase the contact of the sample/lysate P with the sorption filter 362E, in particular its chromatographic/sorbent material, the optional guiding element 362F is arranged within and/or (axially) enclosed by the sorption filter 362E, in particular its chromatographic/sorbent material, preferably centrally and/or equidistantly to the third filter element 362C and the second securing element 362H.

The guiding element 362F is preferably adapted to guide/redirect, in particular center, the flow of the sample/lysate P through the sorption filter 362E, in particular its chromatographic/sorbent material.

In particular, the guiding element 362F is adapted to redirect the flow of the sample/lysate P within the sorption filter 362E away from the housing 361 and/or towards the axis A.

The guiding element 362F is preferably circular and/or embodied as a ring, in particular a sealing ring. With other words, the guiding element 362F preferably reduces the flow area within the sorption filter 362E.

The guiding element 362F is preferably plate-shaped and/or comprises an in particular central passageway/opening.

The guiding element 362F preferably comprises a thickness of more than 0.5 mm or 0.8 mm, in particular of at least essentially 1 mm, and/or of less than 2 mm or 1.5 mm.

The outer diameter of the guiding element 362F is preferably (slightly) larger than the inner diameter of the housing 361, preferably by at least 0.05 mm and/or at most 0.15 mm, preferably by at least essentially 0.1 mm.

The outer diameter of the guiding element 362F is preferably larger than 5 mm or 8 mm and/or smaller than 20 mm or 15 mm, in particular of at least essentially 10 mm.

The inner diameter of the guiding element 362F is preferably larger than 2 mm or 4 mm and/or smaller than 8 mm or 6 mm.

Preferably, the guiding element 362F is made out of plastics, mostly preferred an elastomer.

In the embodiment shown in FIG. 5A, only one guiding element 362F is illustrated. However, it is also possible to arrange a plurality of guiding element 362F within the sorption filter 362E.

The sorption filter 362E is preferably divided into two portions by means of the guiding element 362F.

Preferably, the sorption filter 362E comprises a first portion 362J and a second portion 362K, preferably wherein the guiding element 362F is (axially) arranged between and/or sandwiched by the first portion 362J and the second portion 362K and/or fluidically connects the first portion 362J to the second portion 62K.

The filter arrangement 362 is preferably connected to and/or (axially) held within the housing 361 in a form-fitting and/or force-fitting manner.

To this end, the filter instrument 360, in particular the filter arrangement 362, comprises the securing element(s) 362G, 362H.

It is preferred that the filter instrument 360, in particular the filter arrangement 362, comprises several, here two, securing elements 362G, 362H to secure/hold/fasten the filter arrangement 362, in particular different portions of the filter arrangement 362, at different positions within the housing 361, mostly preferred in a form-fitting and/or force-fitting manner.

The securing elements 362G, 362H are preferably (axially) spaced apart from one another. Preferably, at least one element/component of the filter arrangement 362, here the filter element(s) 362A-362C and the sorption filter 362E, are arranged between the securing elements 362G, 362H.

The first securing element 362G is preferably adapted to maintain the packing density of the filter arrangement 362 and/or to axially secure/hold the (entire) filter arrangement 362, i.e. the (first) filter element 362A, the (second) filter element 362B, the (third) filter element 362C, the sorption filter 362E, the guiding element 362F, and/or the (fourth) filter element 362D, in position.

Preferably, the first securing element 362G is (directly) arranged upstream of the (first) filter element 362A and/or the (second) filter element 362B.

The second securing element 362H is preferably adapted to secure/hold the (fourth) filter element 362D in position.

Preferably, the second securing element 362H is (directly) arranged upstream of the (fourth) filter element 362D and/or between the (fourth) filter element 362D and the sorption filter 362E.

Preferably, the securing elements 362G, 362H and the guiding element 362F are constructed equally. In particular, the first securing element 362G and/or the second securing element 362H comprise(s) one or more features of the guiding element 362F. Therefore, reference is made to the aforementioned description of the guiding element 362F.

Thus, the guiding element 362F preferably also forms a securing element that is arranged within the sorption filter 362E.

However, other solutions are also possible, e.g. wherein the first securing element 362G and/or the second securing element 362H are/is embodied as a securing ring, in particular a snap ring in order to axially secure the filter arrangement 362 and/or single components/elements/portions thereof.

The chamber 365 is preferably defined or delimited by the housing 361 in particular laterally and/or radially and is defined or delimited by the filter arrangement 362, preferably the (first) filter element 362A and/or the (second) filter element 362B, in particular axially.

Preferably, the volume of the chamber 365 can be reduced by actuating the filter instrument 360 and/or by axially moving the plunger 380 towards the outlet 364 or the filter arrangement 362.

Preferably, the filter instrument 360, in particular the chamber 365, has, in the delivery state or in the unactuated state, as shown in FIG. 5A, a maximum volume of more than 2 ml or 4 ml, particularly preferably more than 5 ml, and/or less than 80 ml or 30 ml.

The filter instrument 360, in particular the filter arrangement 362, mostly preferred the sorption filter 362E, is preferably moistened in the delivery state, e.g., with an equilibration buffer. Preferably, the filter instrument 360, in particular the chamber 365, is at least partially filled with an equilibration buffer, in particular for the sorption filter 362E and/or its chromatographic/sorbent material.

The equilibration buffer preferably comprises Tris-HCl, in particular 50 mM thereof, sodium dodecyl sulfate, in particular 0.03125%, and/or sodium azide, in particular 0.05%.

FIG. 5B is a schematic section through the proposed filter instrument 360 according to a second embodiment.

The filter instrument 360 according to the second embodiment is similar to the filter instrument 360 according to the first embodiment. In particular, the filter instrument 360 according to the second embodiment comprises one or more features of the filter instrument 360 according to the first filter instrument 360. Therefore, reference is made to the aforementioned description of the filter instrument 360 according to the first embodiment.

In contrast to the first embodiment, the filter instrument 360 according to the second embodiment does not comprise the (first) filter element 362A, the (third) filter element 362C and the guiding element 362F.

Thus, the (second) filter element 362B comprising or consisting of a cotton, mostly preferred made out of synthetic fiber, is preferably the outermost filter element and/or the filter element which comes first into contact with the sample/lysate P when the sample/lysate P is filled into the filter instrument 360.

The (second) filter element 362B is preferably arranged at an axial end of the filter arrangement 362 and/or at the side of the filter arrangement 362 facing away from the outlet 364 and/or connecting piece 366 and/or towards the inlet 363.

The (second) filter element 362B is preferably arranged (directly) downstream of the first securing element 362G and/or (directly) upstream of the sorption filter 362E. With other words, the (second) filter element 362B is preferably arranged/sandwiched between the first securing element 362G and the sorption filter 362E.

As shown in FIG. 5B, the (second) filter element 362B preferably extends through the first securing element 362G and/or forms a dome protruding out of the first securing element 362G and/or towards the inlet 363.

It has been noted that the (second) filter element 362B—in particular when being arranged at an axial end of the filter arrangement 362 and (directly) upstream of the sorption filter 362E—soaks up the lysate/sample P and/or helps to soak the lysate/sample P through the sorption filter 362E, preferably without the need to push the lysate/sample P through the filter arrangement 362 by means of the plunger 380.

In this way, the sorption filter 362E automatically comes into contact with the sample/lysate P and channeling of the sample/lysate P and/or bypassing of the sorption filter 362E is avoided or at least reduced.

Thus, due to the simplified structure of the filter arrangement 362 of the filter instrument 360 according to the second embodiment, it is possible to omit the guiding element 362F.

In the following, the proposed method is described in greater detail with reference to FIG. 6A to FIG. 6G.

By means of the proposed method, the sample P is preferably pretreated/prepared, in particular lysed and filtered, for a subsequent, in particular molecular-biological test, preferably by means of the cartridge 100 and/or the analyzer 200, in particular such that the sample/lysate P that has been pretreated in this way can be tested or introduced into the cartridge 100, directly and/or without any further treatment steps.

In particular, by means of the proposed method, analytes, particularly preferably nucleic-acid(s) (sequences), are released, extracted, eluted, filtered and/or purified, in particular in order to subsequently detect or identify the analytes or nucleic-acid(s) (sequences), in particular electrochemically, preferably by means of the cartridge 100 and/or the analyzer 200.

Preferably, the proposed method and/or individual method steps of the proposed method, some of the method steps of the proposed method or all of the method steps of the proposed method is/are carried out by means of the proposed kit 300, the receiving instrument 310, the dispensing instrument 320, the lysis tube 340, the treatment apparatus 390 and/or the filter instrument 360.

The proposed method and/or individual method steps of the proposed method, some of the method steps of the proposed method or all of the method steps of the proposed method is/are carried out using the filter instrument 360 according to the first embodiment or the filter instrument 360 according to the second embodiment.

In the following, the proposed method is described using the filter instrument 360 according to the first embodiment. However, also the filter instrument 360 according to the second embodiment can be used.

Preferably, in a first method step (not shown), the sample P is removed, in particular from a human or animal, particularly preferably by means of the receiving instrument 310 or the receiving element 311.

Preferably, a swab, in particular a mucosa swab, more particularly preferably a nasal mucosa swab, is taken for this purpose as receiving instrument 310, sample material preferably being received or absorbed by the receiving element 311.

Preferably, the sample P that has in particular been obtained in this way is pretreated or prepared, in particular by means of the lysis tube 340, the treatment apparatus 390 and/or the filter instrument 360, before the actual test is carried out.

As shown in FIG. 6A, the solvent W, in particular water, is introduced into the lysis tube 340 by means of the dispensing instrument 320, in particular in order to dissolve the treatment agent TA within the lysis tube 340, preferably in a further/second method step.

In a further/third method step, as shown in FIG. 6B, the receiving instrument 310, in particular the receiving element 311, is inserted at least in part into the lysis tube 340, in particular such that the receiving element 311 comes into contact with the dissolved treatment agent TA. Preferably, the dissolved treatment agent TA is stirred up by means of the receiving instrument 310, in particular for at least 10 seconds or 15 seconds.

Optionally, the lysis tube 340 is closed, in particular by means of the cap 342.

In an optional further/fourth method step, the sample P and the treatment agent TA are heat-treated, in particular in order to conduct the lysis, preferably by means of the treatment apparatus 390, as illustrated in FIG. 6C. Preferably, the lysis tube 340 is inserted into a receptacle 391 of the treatment apparatus 390 and, subsequently, heated according to a predefined temperature profile/variation, preferably for at least 10 seconds or 20 seconds.

In this way, the sample P is lysed, i.e. the lysate P is obtained, and/or the analytes, in particular nucleic-acid(s) (sequences) and/or proteins, are extracted from the sample P in the lysis tube 340 and/or eluted by cell disruption.

In an optional further method step, which can be carried out simultaneously, before/during or after the method steps 1 to 3, the container 370 is opened, in particular by removing the closure 372 from the main body 371, and the filter instrument 360 is removed, in particular pulled, out of the container 370, as illustrated in FIG. 6D.

Preferably, in a further, preferably fifth, method step, the sample/lysate P is dispensed to or into the filter instrument 360, in particular out of the lysis tube 340, as shown in FIG. 6E.

For this purpose, the filter instrument 360, in particular the connecting piece 366, is preferably fluidically connected to the cartridge 100, in particular the receiving cavity 104 of the cartridge 100. In particular, the connecting piece 366 of the filter instrument 360 is inserted into the connection 104A of the cartridge 100, as shown in FIG. 3.

In order to receive the sample/lysate P, the cartridge 100 is preferably oriented horizontally and/or laid flat, for example on a table, in particular such that the connection 104A of the cartridge 100 points upwards and/or is accessible from above.

Preferably, the filter instrument 360 is oriented vertically and/or connected to the cartridge 100 or inserted into the connection 104A from above.

Preferably, the sample/lysate P is inserted into the filter instrument 360, in particular by means of the lysis tube 340, particularly preferably through the inlet 363 and/or from above, in particular such that the sample/lysate P collects at the bottom or on the filter arrangement 362.

As shown in FIG. 6F, the sample/lysate P is (automatically) soaked in/through the filter arrangement 362, in particular by means of the (second) filter element 362B and/or such that the sorption filter 362E comes into contact with the sample/lysate P.

It is preferred that the sorption filter 362E is completely soaked with the sample/lysate P and/or that the sample/lysate P interacts with the sorption filter 362E for a predefined contact time, in particular without using the plunger 380 and/or causing additional pressure by means of the plunger 380.

To this end, after inserting the sample/lysate P into the filter instrument 360, the plunger 380 is only inserted when a predefined contact time has passed, e.g., more than 30 seconds and/or less than 5 minutes, in particular when 1 minute has passed since insertion of the sample/lysate P.

Subsequently and/or when the predefined contact time has passed, in particular in a further/sixth method step, the sample/lysate P is depressed to or into the cartridge 100, in particular using the plunger 380.

The plunger 380, preferably its shaft 382 or head 381, is preferably inserted into the filter instrument 360 until the stop 384 abuts the filter instrument 360, in particular its holder/collar 367, mostly preferred axially, and/or until the end position of the plunger 380 is reached.

By moving the plunger 380, preferably its shaft 382 or head 381, towards the filter arrangement 362 and/or downwards, the pressure in the chamber 365 is preferably increased, in particular until the pressure resistance of the filter arrangement 362 is overcome, the sample/lysate P is pushed through the filter arrangement 362, into the cartridge 100 and/or the sample/lysate P is filtered/purified by means of the filter arrangement 362.

Preferably, the pressure generated in the filter instrument 360 is limited by means of the stop 384.

In particular since the air between the plunger 380, in particular its head 381, and the filter arrangement 362 acts as an air cushion/spring and/or causes a delay in dispensing the sample/lysate P to the cartridge 100, after reaching the end position of the plunger 380 and/or after the stop 384 abuts the filter instrument 360, in particular its holder/collar 367, a predefined waiting time, e.g., of more than 10 seconds and/or less than 30 seconds, has to elapse before the delivery of the sample/lysate P is completed and/or the filter instrument 360 is to be separated from the cartridge 100, in particular in order to guarantee that a sufficient/predefined amount of the sample/lysate P is dispensed/delivered into the cartridge 100.

As already mentioned, the plunger 380 is preferably equipped with the locking part 385 and the filter instrument 360 is preferably equipped with a matching part 368, or vice versa, in order to secure the axial position of the plunger 380 relative to the filter instrument 360 when the plunger 380 reaches the end position and/or when the stop 384 abuts the filter instrument 360.

Preferably, the plunger 380, in particular its shaft 382 or head 381, is inserted into the filter instrument 360 until the locking part 385 engages the matching part 368, mostly preferred in a form-fitting matter and/or by means of a clip-on connection, in particular such that the axial position of the plunger 380 relative to the filter instrument 360 is secured.

With other words, the plunger 380 is automatically clicked into place after being inserted into the filter instrument 360 and/or when reaching the end position, preferably such that the plunger 380 cannot be pulled or pushed out of the filter instrument 360 once the end position is reached.

In this way, the axial position of the plunger 380 is maintained/secured during dispensing of the sample/lysate P, thereby facilitating the handling of the plunger 380 and the filter instrument 360 and reducing the risk of misapplication. Further, the plunger 380 and the filter instrument 360 can be disposed as one unit and/or without the risk of leakage.

The sample/lysate P is therefore preferably filtered, in particular by means of the integrated filter elements 362A to 362D, and/or purified, in particular by means of the sorption filter 362E, while or immediately before the sample/lysate P is dispensed to the cartridge 100, the pressure required for filtration preferably being created in the chamber 365 by actuating the filter instrument 360 or the plunger 380.

Preferably, the filter instrument 360 is then separated from the cartridge 100, in particular with the plunger 380 being secured/connected thereto in a form-fitting manner.

After the sample/lysate P has been received, the cartridge 100 is preferably closed in a liquid-tight and/or gas-tight manner by means of the closure element 130, in particular such that the cartridge 100 together with the sample/lysate P can be inserted into the analyzer 200 for the subsequent test.

In particular, the present invention relates also to any one of the following aspects which can be realized independently or in any combination, also in combination with any aspects described above and in the following aspects:

1. Filter instrument (360) for filtering an in particular biological sample (P), the filter instrument (360) comprising a preferably cylindrical housing (361) and a filter arrangement (362) within the housing (361),
wherein the filter arrangement (362) comprises a sorption filter (362E) for separation of biopolymers, in particular nucleic acids, characterized in that the filter arrangement (362) comprises a guiding element (362F) arranged within the sorption filter (362E), and/or in that the filter arrangement (362) comprises at least two securing elements (362G, 362H) located at different positions within the housing (361) to hold the filter arrangement (362).
2. Filter instrument according to aspect 1, characterized in that the sorption filter (362E) comprises a chromatographic/sorbent material, in particular a silica gel, and/or is powdery/granular.
3. Filter instrument according to aspect 1 or 2, characterized in that the sorption filter (362E) is adapted to—in particular chromatographically—separate nucleic acids, in particular DNA and/or RNA, from other biopolymers, in particular proteins.
4. Filter instrument according to any one of the preceding aspects, characterized in that the guiding element (362F) is adapted to guide, in particular center, the flow through the sorption filter (362E) and/or in that the guiding element (362F) reduces the flow area within the sorption filter (362E).
5. Filter instrument according to any one of the preceding aspects, characterized in that the sorption filter (362E) is divided into two—in particular equally sized—portions (362J, 362K) by means of the guiding element (362F).
6. Filter instrument according to any one of the preceding aspects, characterized in that the guiding element (362F) is circular and/or embodied as a sealing ring.
7. Filter instrument according to any one of the preceding aspects, characterized in that each securing element (362G, 362H) is circular and/or embodied as a sealing ring.
8. Filter instrument according to any one of the preceding aspects, characterized in that the filter arrangement (362) comprises a filter element (362A, 362B, 362C, 362D), wherein the filter element (362A, 362B, 362C, 362D) is embodied as a preferably plate-shaped frit.
9. Kit (300) for pretreating an in particular biological sample (P) for an in particular molecular-biological test of the sample (P), the kit (300) comprising a filter instrument (360) for filtering the sample (P) and a plunger (380) to push the sample (P) through the filter instrument (360), characterized in that the filter instrument (360) is designed according to any one of the preceding aspects, and/or in that the plunger (380) comprises a stop (384) to prevent complete insertion of the plunger (380) into the filter instrument (360).
10. Kit according to aspect 9, characterized in that the kit (300) comprises a container (370) containing the filter instrument (360) in order to protect the filter arrangement (362) from drying out.
11. Kit according to aspect 9 or 10, characterized in that kit (300), in particular the container (370), mostly preferred its closure (372), comprises a humidity buffer (373) in order to moderate changes in relative humidity, preferably wherein the humidity buffer (373) comprises silica gel.
12. Kit according to any one of the aspects 9 to 11, characterized in that the kit (300) comprises a receiving instrument (310) for receiving the sample (P), a lysis tube (340) for extracting the sample (P) from the receiving instrument (310), a dispensing instrument (320) and/or a preferably dry treatment agent (TA) for treating the sample (P), the treatment agent (TA) preferably comprising a lysis buffer and/or enzymes for lysis of the sample (P).
13. Method for pretreating an in particular biological sample (P), preferably an oral fluid sample (P), for an in particular molecular-biological test of the sample (P) within a cartridge (100), comprising the steps of:
lysis of the sample (P) by means of a treatment agent (TA) in order to obtain a lysate (P), inserting the lysate (P) into a filter instrument (360), and
pushing the lysate (P) through a filter arrangement (362) arranged in the filter instrument (360),
wherein the lysate (P) is filtered and nucleic acids, in particular DNA and/or RNA, are—in particular chromatographically—separated from other biopolymers, in particular proteins, within the filter arrangement (362), characterized in that the flow of the lysate (P) is centered within the filter arrangement (362).
14. Method according to aspect 13, wherein the volume pushed through the filter arrangement (362) and/or the pressure generated in the filter instrument (360) is limited by means of a stop (384) of a plunger (380).
15. Method according to aspect 13 or 14, wherein the filter instrument (360) is designed according to any one of the aspects 1 to 8.
Individual aspects and features of the present invention and individual method steps and/or method variants may be implemented independently from one another, but also in any desired combination and/or order.

What is claimed is:

1. A filter instrument for filtering a sample, comprising:
a housing and
a filter arrangement within the housing,
wherein the filter arrangement comprises a sorption filter for separation of biopolymers, a filter element, and at least two securing elements located at different positions within the housing to hold the filter arrangement.

2. The filter instrument according to claim 1, wherein the sorption filter comprises a material from the group consisting of at least one of chromatographic, sorbent, powdery, granular and a silica gel.

3. The filter instrument according to claim 1, wherein the sorption filter is adapted to separate nucleic acids from other biopolymers.

4. The filter instrument according to claim 1, wherein each of the at least two securing elements is at least one of circular, ring-shaped and a sealing ring.

5. The filter instrument according to claim 1, wherein the at least two securing elements comprises a first securing element and a second securing element.

6. The filter instrument according to claim 1, wherein the filter element comprises a frit.

7. The filter instrument according to claim 5, wherein the filter element comprises a frit and wherein the second securing element is directly arranged at least one of upstream of the filter element or between the filter element and the sorption filter.

8. The filter instrument according to claim 1, wherein the filter element comprises or consists of cotton.

9. The filter instrument according to claim 5, wherein the filter element comprises or consists of cotton, and wherein the first securing element is directly arranged upstream of the filter element.

10. A kit for pretreating a sample for a test of the sample, comprising:
a filter instrument for filtering the sample,
a plunger to push the sample through the filter instrument, and
at least one of the following features:
the filter instrument comprising a housing and a filter arrangement within the housing, wherein the filter arrangement comprises a sorption filter for separation of biopolymers, a filter element, and at least two securing elements located at different positions within the housing to hold the filter arrangement, or
the plunger comprising a stop to prevent complete insertion of the plunger into the filter instrument.

11. The kit according to claim 10, wherein the kit comprises a container containing the filter instrument in order to protect the filter arrangement from drying out.

12. The kit according to claim 10, wherein the kit or a container thereof comprises a humidity buffer in order to moderate changes in relative humidity.

13. The kit according to claim 10, wherein the plunger or the filter instrument comprises a locking part to secure the axial position of the plunger relative to the filter instrument.

14. The kit according to claim 13, wherein the plunger or the filter instrument comprises a matching part, wherein the locking part is adapted to engage the matching part.

15. The kit according to claim 10, wherein the kit further comprises at least one of a receiving instrument for receiving the sample, a lysis tube for extracting the sample from the receiving instrument, a dispensing instrument or a treatment agent for treating the sample.

16. A method for pretreating a sample for testing of the sample within a cartridge, comprising the steps of:
analyzing the sample by means of a treatment agent in order to obtain a lysate,
inserting the lysate into a filter instrument, and
pushing the lysate through a filter arrangement arranged in the filter instrument by means of a plunger,
filtering the lysate and separating nucleic acids from other biopolymers within the filter arrangement, and
securing the axial position of the plunger relative to the filter instrument after insertion of the plunger into the filter instrument.

17. The method according to claim 16, wherein at least one of a volume pushed through the filter arrangement or a pressure generated in the filter instrument is limited by means of a stop of the plunger.

18. The method according to claim 16, wherein the plunger is automatically clicked into place at least one of after being inserted into the filter instrument or when reaching an end position.

19. The method according to claim 16, wherein the plunger and the filter instrument are at least one of connected to one another in a form-fit manner or connected to one another by means of a clip-on connection.

20. The method according to claim 16, wherein the filter instrument comprises a housing and a filter arrangement within the housing, the filter arrangement comprising a sorption filter for separation of biopolymers, a filter element, and at least two securing elements located at different positions within the housing to hold the filter arrangement.
